(19) United States
(12) Patent Application Publication
(10) Pub. No.: US 2009/0012182 A1
(43) Pub. Date: Jan. 8, 2009

(54) Crystal forms of O-desmethylvenlafaxine succinate

(76) Inventors: Alexandr Jegorov, Dobra Voda (CZ); Eli Lancry, Modiin (IL); Anna Balanov, Rehovot (IL)

Correspondence Address: KENYON & KENYON LLP, ONE BROADWAY, NEW YORK, NY, 10004, US

(21) Appl. No.: 12/214,156

(22) Filed: Jun. 16, 2008

Related U.S. Application Data
(60) Provisional application No. 60/934,740, filed on Jun. 15, 2007.
(60) Provisional application No. 61/035,571, filed on Mar. 11, 2008.
(60) Provisional application No. 61/041,678, filed on Apr. 2, 2008.

(51) Int. Cl. A61K 31/137; C07C 211/27; A61P 25/24

(52) U.S. Cl. 514/654; 564/374

(57) ABSTRACT

Provided are crystalline forms of O-desmethylvenlafaxine succinate, methods for their preparation, and pharmaceutical composition thereof.

### CROSS REFERENCE TO RELATED APPLICATIONS

The present invention claims the benefit of the following U.S. Provisional Patent Application Nos. 60/934,740, filed Jun. 15, 2007; 61/035,571, filed Mar. 11, 2008; and 61/041,678, filed Apr. 2, 2008. The contents of these applications are incorporated herein by reference.

### FIELD OF INVENTION

The present invention is directed to crystalline O-desmethylvenlafaxine succinate and method of its preparation.

### BACKGROUND OF THE INVENTION

Venlafaxine, (±)-1-[2-(Dimethylamino)-1-(4-ethyoxyphenyl)ethyl]cyclo-hexanol, having the following formula

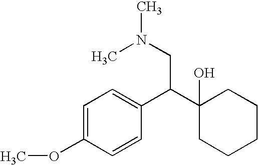

is the first of a class of anti-depressants. Venlafaxine acts by inhibiting re-uptake of norepinephrine and serotonin, and is an alternative to the tricyclic anti-depressants and selective re-uptake inhibitors.

O-desmethylvenlafaxine (ODV), chemically named 4-[2-(dimethylamino)-1-(1-hydroxycyclohexyl)ethyl]phenol having the following formula,

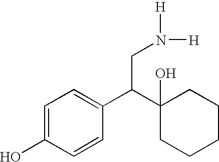

is a major metabolite of venlafaxine and has been shown to inhibit norepinephrine and serotonin uptake. Klamerus, K. J. et al., “Introduction of the Composite Parameter to the Pharmacokinetics of Venlafaxine and its Active O-Desmethyl Metabolite”, J. Clin. Phavmacol. 32:716-724 (1992).

O-desmethylvenlafaxine and processes for the preparation thereof are described in U.S. Pat. Nos. 6,197,828 and 6,689,912, and in US 2005/0197392, which are incorporated herein by reference.

Venlafaxine base can be used as a starting material in the preparation of O-desmethylvenlafaxine, as demonstrated in U.S. Pat. No. 6,689,912, U.S. Pat. No. 6,197,828, WO 03/048104 and US 2005/0197392.

O-desmethylvenlafaxine succinate, its polymorphs, including forms I, II, III, and IV, and processes for preparation thereof are described in U.S. Pat. No. 6,673,838. Crystalline forms of O-desmethylvenlafaxine succinate are also disclosed in WO 2008/047167 and WO 2008/017886.

Polymorphism, the occurrence of different crystal forms, is a property of some molecules and molecular complexes. A single molecule, like O-desmethylvenlafaxine, may give rise to a variety of crystalline forms having distinct crystal structures and physical properties like melting point, x-ray diffraction pattern, infrared absorption fingerprint, FTIR spectrum, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

There is a need in the art for polymorphic forms of O-desmethylvenlafaxine succinate.

### SUMMARY OF THE INVENTION

In one embodiment, the present invention provides crystalline O-desmethylvenlafaxine succinate (ODV succinate), characterized by data selected from the group consisting of: an X-ray powder diffraction having peaks at about: 5.2, 10.3, 16.7 and 25.8±0.2 degrees two theta; a PXRD spectrum as depicted in FIG. 1; a solid state ¹³C-NMR spectrum as depicted in FIG. 2; and a combination thereof.

In another embodiment, the present invention provides a hydrate form of O-desmethylvenlafaxine succinate. Preferably, the O-desmethylvenlafaxine succinate hydrate is a monohydrate, having a water content of about 4% to about 5.5%, preferably 4.5% to about 5%, more preferably about 4.7%, determined by KF.

In another embodiment, the present invention provides a method of preparing the crystalline form of O-desmethylvenlafaxine succinate described above comprising: suspending ODV succinate in a solvent selected from a C₃₋₆ ketone, preferably methylethylketone (MEK), or a C₄₋₈ ether, preferably t-butyl methyl ether.

In one embodiment, the present invention provides crystalline ODV succinate, characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof.

In another embodiment, the present invention provides a method of preparing the crystalline form of O-desmethylvenlafaxine succinate described above comprising: combining O-desmethylvenlafaxine, a C₁₋₄ alcohol, preferably ethanol, a C₆₋₈ aliphatic or aromatic hydrocarbon, preferably toluene and succinic acid, and precipitating the crystalline ODV succinate out of the reaction mixture.

In another embodiment, the present invention provides pharmaceutical compositions comprising the above crystalline forms of O-desmethylvenlafaxine succinate and at least one pharmaceutically acceptable excipient.

In another embodiment, the present invention provides a process for preparing the above pharmaceutical composition comprising combining any of the crystalline forms O-desmethylvenlafaxine succinate of the present invention and at least one pharmaceutically acceptable excipient.

In another embodiment, the present invention provides a method of inhibiting re-uptake of norepinephrine and serotonin in a patient comprising administering to a patient in need thereof a therapeutically effective amount of the above crystalline forms O-desmethylvenlafaxine succinate.

In another embodiment, the present invention provides a method of treating a patient comprising administering to a patient in need thereof a therapeutically effective amount of the above crystalline form of O-desmethylvenlafaxine succinate.

### BRIEF DESCRIPTION OF THE FIGURES

FIG. 1 shows a PXRD pattern of crystalline O-desmethylvenlafaxine succinate obtained in example 1.

FIG. 2 shows a solid state ¹³C-NMR of crystalline O-desmethylvenlafaxine succinate obtained in example 1.

FIG. 3 shows a PXRD pattern of crystalline O-desmethylvenlafaxine succinate obtained in example 2.

### DETAILED DESCRIPTION OF THE INVENTION

As used herein the term “room temperature” refers to the ambient temperature of a typical laboratory, which is usually about that of Standard Temperature and Pressure.

As used herein the term “solidification” refers to the process of transforming a composition from a liquid or molten phase into a solid phase.

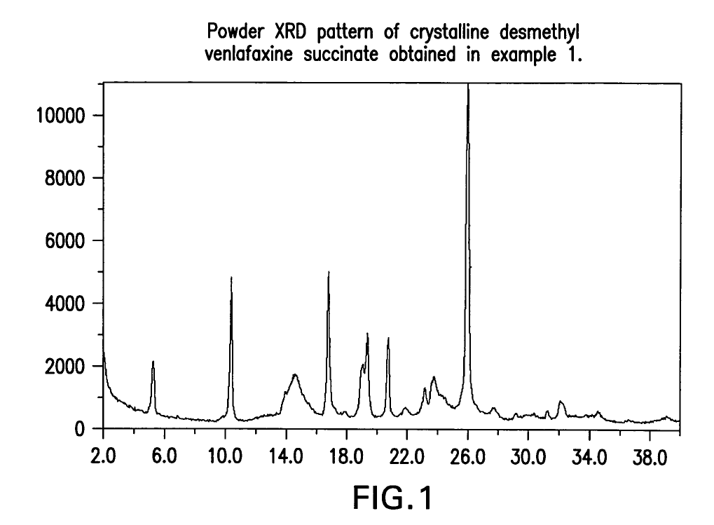

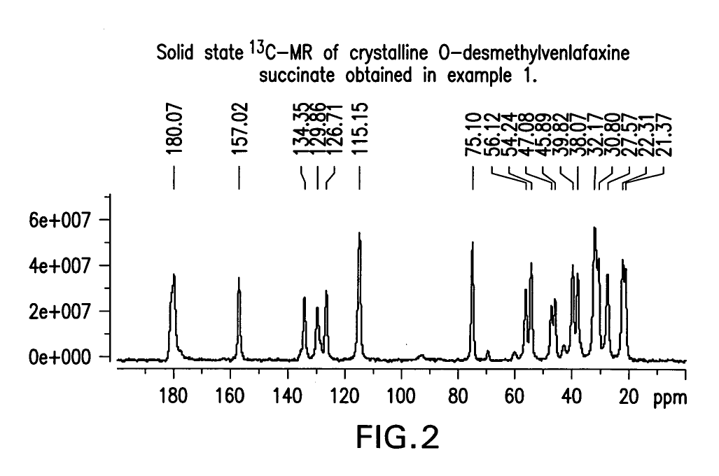

In one embodiment, the present invention provides a crystalline form of O-desmethylvenlafaxine succinate (ODV succinate), characterized by data selected from the group consisting of at least one of: an X-ray powder diffraction having peaks at about: 5.2, 10.3, 16.7 and 25.8±0.2 degrees two theta; a PXRD spectrum as depicted in FIG. 1; a solid state ¹³C-NMR spectrum as depicted in FIG. 2; and a combination thereof.

The above crystalline form of ODV succinate can be further characterized by an X-ray powder diffraction pattern having additional peaks at about 14.4, 20.6 and 31.9±0.2 degrees two theta.

In yet another embodiment, the present invention provides a hydrate form of O-desmethylvenlafaxine succinate. Preferably, the O-desmethylvenlafaxine succinate hydrate is a monohydrate, having a water content of about 4% to about 5.5%, preferably 4.5% to about 5%, more preferably about 4.7%, determined by KF. Preferably, the hydrate form of O-desmethylvenlafaxine succinate is characterized by data selected from the group consisting of: an X-ray powder diffraction having peaks at about: 5.2, 10.3, 16.7 and 25.8±0.2 degrees two theta; a PXRD spectrum as depicted in FIG. 1; a solid state ¹³C-NMR spectrum as depicted in FIG. 2; and a combination thereof.

In another embodiment, the present invention provides a method of preparing the crystalline form of O-desmethylvenlafaxine succinate described above comprising: suspending ODV succinate in a C₃₋₆ ketone, preferably methylethylketone (MEK), or a C₄₋₈ ether, preferably t-butyl methyl ether, to obtain the above crystalline ODV. The ODV succinate starting material can be in any crystalline or the amorphous form. Optionally, the ODV succinate is at least partially melted and then solidified, prior to suspending it. As used herein the term partially melted refers to ODV succinate in which individual particles of substance have melted but wherein a complete liquid melt was not formed. Preferably the process of preparing the above crystalline O-desmethylvenlafaxine succinate comprises a) suspending O-desmethylvenlafaxine succinate in a solvent selected from a C₃₋₆ ketone and a C₄₋₈ ether; b) heating the obtained suspension; and c) cooling the heated suspension to obtain the crystalline O-desmethylvenlafaxine succinate. Preferably the C₃₋₆ ketone is methylethylketone (MEK), the C₄₋₈ ether is preferably t-butyl methyl ether. More preferably, the ODV succinate in step a) is provided by a process comprising: a) partially melting crystalline O-desmethylvenlafaxine succinate; and b) solidifying the partially molten O-desmethylvenlafaxine succinate.

The above partially melted ODV succinate used as starting material can be obtained, for example, by heating ODV succinate under reduced pressure. Preferably, this ODV succinate is a mixture of form I and II. Form I and Form II of ODV succinate can be prepared according to any method known in the art, i.e; the method disclosed in U.S. Pat. No. 6,673,838. As described therein Form I of O-desmethylvenlafaxine succinate may be prepared by dissolving the free base of O-desmethylvenlafaxine and succinic acid in aqueous acetone and cooling the resulting solution to obtain crystalline O-desmethylvenlafaxine succinate Form I. Further, as described therein crystalline Form II of O-desmethylvenlafaxine may be prepared by slowly cooling of either saturated acetone or 95:5 ethanol:water solutions of crystalline ODV succinate form I.

Preferably, the reduced pressure in the process of partially melting ODV succinate is less than about 100 mBar, more preferably less than about 10 mBar, even more preferably about 1 mBar. Preferably, heating therein is to temperature of about 125° C. to about 150° C., more preferably to about 130° C. to about 140° C., even more preferably to about 135° C. Preferably, heating is for a period of about 1 hour to about 4 hours, more preferably of about 2 hours to about 3 hours, even more preferably of about 2 hrs. Dependent on the temperature, this heating provides a melted or partially melted mixture.

Preferably, the melted mixture undergoes solidification, when cooled to below 100° C.

The solidified ODV succinate is then combined with a solvent selected from a C₃₋₆ ketone, preferably methylethylketone (MEK), or a C₄₋₈ ether, preferably t-butyl methyl ether to obtain a suspension.

The ratio of O-desmethylvenlafaxine succinate to solvent is preferably about 1:10 to about 1:30 (w/v).

Preferably, the suspension is heated to a temperature of about 50° C. to about reflux temperature, more preferably of about 75° C. to about reflux temperature, even more preferably to about reflux temperature. Preferably, heating the suspension is for a period of about 30 minutes to about 2 hours, more preferably for about 1 hour to about 90 minutes, even more preferably for about 1 hour.

The heated suspension is then cooled, preferably to a temperature of about 15° C. to about 30° C., more preferably of about 15° C. to about 25° C., even more preferably of about 20° C.

The cooling is preferably done over a period of about 1 hour to about 12 hours, more preferably for about 2 hours to about 6 hours, even more preferably for about 3 hours.

Preferably, after about 3 hours of cooling, most of the dissolved matter crystallizes.

The obtained crystalline form may then be recovered by any method known in the art, such as filtering and drying. Optionally, the drying is under a nitrogen stream.

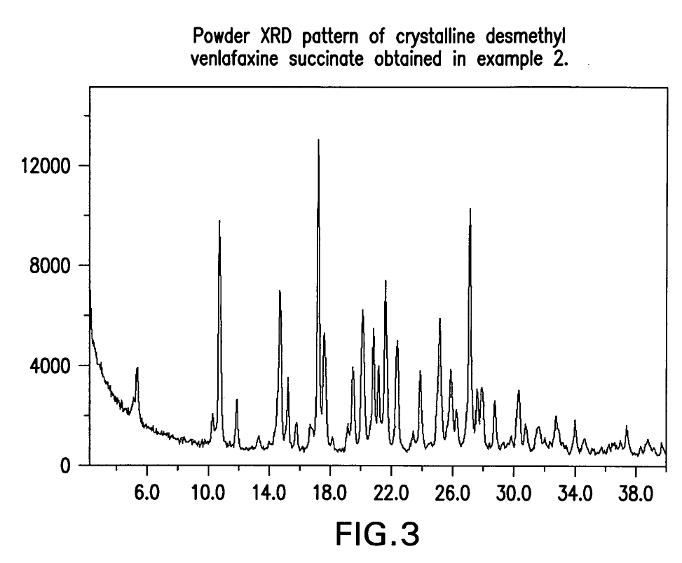

In one embodiment, the present invention provides crystalline ODV succinate, characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof. Preferably, the crystalline ODV succinate is characterized by an X-ray powder diffraction having peaks at about 5.3, 10.7, 14.6, 17.2, 17.6, 21.6, 25.1, and 27.1±0.2 degrees two theta.

In another embodiment, the present invention provides a method of preparing the crystalline form of O-desmethylvenlafaxine succinate described above comprising: combining O-desmethylvenlafaxine, a C₁₋₄ alkyl alcohol, preferably ethanol, a C₆₋₈ aliphatic or aromoatic hydrocarbon, preferably hexane or toluene and succinic acid to precipitate the crystalline ODV succinate out of the reaction mixture.

The O-desmethylvenlafaxine starting material is preferably in a base form, preferably crystalline O-desmethylvenlafaxine free base Form A characterized by an X-ray powder diffraction having peaks at about 12.1, 13.2, 15.9, 20.4±0.2 degrees two theta.

The ratio of ethanol to toluene is preferably about 1:10 to about 1:4 (v/v), preferably about 2:9. The ratio of O-desmethylvenlafaxine succinate to the C₁₋₄ alkyl alcohol, preferably ethanol is preferably about 1:2 to about 2:1, preferably about 1:2 (w/v).

The reagents of the reaction mixture are mixed under conditions adequate to form a solution. Preferably, the reaction mixture is heated to reflux to promote dissolution. The solution is then cooled in order to promote precipitation.

In one specific embodiment, toluene or hexane are combined with a solution of succinic acid, O-desmethylvenlafaxine and ethanol. This solution is obtained by suspending ODV base in ethanol at a temperature of about 70° C. to about reflux temperature, preferably at about reflux temperature, and adding succinic acid to the suspension.

Preferably, the toluene or hexane are added to this solution in a drop wise manner.

Typically, following the addition of toluene or hexane, the reaction mixture is cooled to about 15° C.-30° C., preferably to about 15° C.-25° C., more preferably to about room temperature, and further maintained at such temperature for a sufficient period of time to obtain the ODV succinate crystalline form. The cooled reaction mixture is preferably maintained at the cooling temperature for a period of about 12 hours to about 36 hours, more preferably for about 16 hours to about 24 hours, most preferably for about 16 hours.

The obtained crystalline form may then be recovered by any conventional method.

In another embodiment, the present invention provides pharmaceutical compositions comprising the above crystalline forms O-desmethylvenlafaxine succinate and at least one pharmaceutically acceptable excipient.

In another embodiment, the present invention provides a process for preparing the above pharmaceutical composition comprising combining any of the crystalline forms O-desmethylvenlafaxine succinate of the present invention and at least one pharmaceutically acceptable excipient.

In another embodiment, the present invention provides a method of inhibiting re-uptake of norepinephrine and serotonin in a patient comprising administering to a patient in need thereof a therapeutically effective amount of the above crystalline forms O-desmethylvenlafaxine succinate.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups, and suspensions. Suitable forms of parenteral administration include an aqueous or non-aqueous solution or emulsion, while for rectal administration, suitable forms for administration include suppositories with hydrophilic or hydrophobic vehicle. For topical administration, the invention provides suitable transdermal delivery systems known in the art, and for nasal delivery, there are provided suitable aerosol delivery systems known in the art.

In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier such as water, vegetable oil, alcohol, polyethylene glycol, propylene glycol or glycerin. In such suspension the solid excipients may be either in solution or suspended in the liquid carrier. The active ingredient retains its crystalline structure in such liquid pharmaceutical compositions.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate, or sodium acetate.

Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention may be a capsule containing the composition, preferably a powdered or granulated solid composition of the invention, within either a hard or soft shell. The shell may be made from gelatin, and, optionally, contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted or other excipients may be added prior to tableting, such as a glidant and/or a lubricant.

A tableting composition may be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

In another embodiment, the present invention provides a method of treating a patient comprising administering to a patient in need thereof a therapeutically effective amount of the above crystalline form of O-desmethylvenlafaxine succinate. Preferably, the patient suffers from a condition which may be treated with a norepinephrine or a serotonin re-uptake inhibitor. Such patient may be suffering from depression.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### XRD

ARL (Scintag) X-Ray powder diffractometer model X'TRA, Cu-tube, solid state detector. Copper radiation of 1.5418 Å was used

Sample holder: a round standard aluminum sample holder with round zero background quartz plate.

### Scanning Parameters:

All range 2-40 deg. two theta, Continuous scan, step: 0.05 deg., cnt time: 1.0 sec
Slow scan range: 2-40 deg. two theta, step scan, step: 0.02 deg., cnt time: 20 sec

### KF

Water content was determined by Karl Fischer titrator TITRANDO 841, software Tiamo 1.1 (Metrohm). Solution used for determination: Hydranal Composite 2 (Riedel de Haen). Sampling: 100.00 mg, 2 repeats.

### Solid State ¹³C-NMR

Solid state ¹³C-NMR spectra were measured at 125 MHz using Bruker Avance 500 WB/US NMR spectrometer (Karlsruhe, Germany, 2003) at magic angle spinning (MAS) frequency 11 kHz. Finely powdered sample was placed into the 4 mm ZrO₂ rotors and standard CPMAS pulse program was used. During the acquisition of the data a high-power dipolar decoupling TPPM (two-pulse phase-modulated) was applied. The phase modulation angle was 15°, and the flip-pulse length was 4.8 μs. Applied nutation frequency of B1(1H) field was ω₁/2π=89.3 kHz. Nutation frequency of B1(13C) and B1(1H) fields during cross-polarization was ω₁/2π=62.5 kHz and repetition delay was 4 s. The 13C scale was calibrated with glycine as external standard (176.03 ppm—low-field carbonyl signal).

### Example 1

A mixture of O-desmethylvenlafaxine succinate forms I and II (25 gr) was placed in a vacuum oven at 135° C. under 1 mBar for 2 hours. The weight of sample was monitored providing the weight loss 1.225 mg. Sample was partially melted and solidified at ambient temperature. 3.5 gr of the solidified mater was placed in a glass flask, methylethylketone was added (100 ml) and the suspension was heated under reflux temperature for 1 h. During the heating, the sample was partially dissolved. The suspension was allowed to cool to 20° C. Within about 3 h, the majority of dissolved matter crystallized out. The insoluble matter was filtered and dried by a stream of nitrogen, yield 3.1 g. Water content determined by KF was about 4.7%. The obtained crystalline O-desmethylvenlafaxine succinate (ODV succinate), is characterized by data selected from the group consisting of at least one of: an X-ray powder diffraction having peaks at about: 5.2, 10.3, 16.7 and 25.8±0.2 degrees two theta; a PXRD spectrum as depicted in FIG. 1; a solid state ¹³C-NMR spectrum as depicted in FIG. 2; and a combination thereof.

### Example 2

ODV base Form A (1 g) was suspended in ethanol (2 ml) at reflux. Succinic acid (0.49 g) was added. The mixture became clear solution. Toluene (9 ml) was slowly added and the mixture was cooled and stirred at 20° C. for 16 h. Slow crystallization occurred. The precipitate was filtered and measured by XRD to obtain crystalline ODV Succinate. The obtained crystalline ODV succinate, is characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof.

### Example 3

ODV base Form A (1 g) was suspended in ethanol (3 ml) at reflux. Succinic acid (0.49 g) was added. The mixture became a clear solution. Hexane (5 ml) was slowly added and the mixture was cooled to 20° C. Some oily material began to precipitate and after 4 hours stirring at this temperature, hexane (15 ml) was added. The mixture was stirred at this temperature for 18 hours. The precipitate that was formed was filtered and measured by XRD to obtain crystalline ODV Succinate. The obtained crystalline ODV succinate, is characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof.

### Example 4

ODV base (1 g) was suspended in ethanol (3 ml) at reflux. Succinic acid (0.49 g) was added. The mixture became a clear solution. Hexane (5 ml) was slowly added and the mixture was cooled and stirred at 20° C. for 16 h. Oil precipitation was observed. Additional portion of hexane (15 mL) was added. Slow crystallization of the oil occurred. The precipitate was filtered and measured by XRD to obtain crystalline ODV Succinate. The obtained crystalline ODV succinate, is characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof.

What is claimed is:

1. Crystalline form of O-desmethylvenlafaxine succinate (ODV succinate), characterized by data selected from the group consisting of: an X-ray powder diffraction having peaks at about: 5.2, 10.3, 16.7 and 25.8±0.2 degrees two theta; a PXRD spectrum as depicted in FIG. 1; a solid state ¹³C-NMR spectrum as depicted in FIG. 2; and a combination thereof.

2. The crystalline form of O-desmethylvenlafaxine succinate of claim 1, characterized by an X-ray diffraction pattern having peaks at about 5.2, 10.3, 16.7 and 25.8±0.2 degrees two theta.

3. The crystalline form of O-desmethylvenlafaxine succinate of claim 1, characterized by a PXRD spectrum as depicted in FIG. 1.

4. The crystalline form of O-desmethylvenlafaxine succinate of claim 1, characterized by a solid state ¹³C-NMR spectrum as depicted in FIG. 2.

5. The crystalline form of O-desmethylvenlafaxine succinate of claim 1, further characterized by an X-ray powder diffraction pattern having additional peaks at about 14.4, 20.6 and 31.9±0.2 degrees two theta.

6. The crystalline form of O-desmethylvenlafaxine succinate of claim 1, wherein the crystalline forms is a hydrate.

7. The crystalline form of O-desmethylvenlafaxine succinate of claim 6, wherein the hydrate has a water content of about 4% to about 5.5% as determined by KF.

8. The crystalline form of O-desmethylvenlafaxine succinate of claim 7, wherein the hydrate has a water content of about 4.7% as determined by KF.

9. The crystalline form of O-desmethylvenlafaxine succinate of claim 6, wherein the hydrate is a monohydrate.

10. A method of preparing the crystalline form of O-desmethylvenlafaxine succinate of claim 1 comprising: suspending ODV succinate in a solvent selected from a C₃₋₆ ketone or a C₄₋₈ ether, to obtain the above crystalline ODV.

11. The method of claim 10, wherein the C₃₋₆ ketone is methylethylketone (MEK) and the C₄₋₈ ether is t-butyl methyl ether.

12. The method of claim 10, wherein the ODV succinate is at least partially melted and then solidified, prior to suspending it.

13. The method of claim 10 comprising a) suspending O-desmethylvenlafaxine in a solvent selected from a C₃₋₆ ketone or a C₄₋₈ ether; b) heating the obtained suspension; and c) cooling the heated suspension to obtain the crystalline O-desmethylvenlafaxine.

14. The method of claim 13, wherein the C₃₋₆ ketone is methylethylketone (MEK) and the C₄₋₈ ether is t-butyl methyl ether.

15. The method of any of claims 10 or 13, further comprising a) at least partially melting crystalline O-desmethylvenlafaxine; and b) solidifying the at least partially molten O-desmethylvenlafaxine prior to suspending O-desmethylvenlafaxine in the solvent selected from a C₃₋₆ ketone or a C₄₋₈ ether.

16. The method of claim 15, wherein at least partially melting ODV succinate comprises heating ODV succinate under a pressure of less than one atmosphere.

17. The method of claim 16, wherein heating ODV succinate under reduced pressure comprises heating a mixture of ODV succinate form I and II under a pressure of less than one atmosphere.

18. The method of claim 16, wherein the pressure is less than about 100 mBar.

19. The method of claim 18, wherein the pressure is about 1 mBar.

20. The method of claim 16, wherein heating is to temperature of about 125° C. to about 150° C.

21. The method of claim 20, wherein the temperature is about 135° C.

22. The method of claim 16, wherein heating is conducted for a period of about 1 hour to about 4 hours, providing an at least partially melted mixture.

23. The method of claim 15, wherein the partially melted mixture undergoes solidification when cooled to less than about 100° C.

24. The method of claim 13, wherein the suspension is heated to about 50° C. to about reflux temperature.

25. The method of claim 24, wherein the suspension is heated to about reflux.

26. The method of claim 24, wherein the suspension is heated for a period of about 30 minutes to about 2 hours.

27. The method of claim 26, wherein the suspension is heated for a period of about 1 hour.

28. The method of claim 13, wherein the heated suspension is cooled to a temperature of about 15° C. to about 30° C.

29. The method of claim 28, wherein the heated suspension is cooled to about 20° C.

30. The method of claim 28, wherein cooling is conducted over a period of about 1 hour to about 12 hours.

31. The method of claim 30, wherein cooling is conducted over a period of about 3 hours.

32. Crystalline ODV succinate, characterized by data selected from the group consisting of: an X-ray powder diffraction having peaks at about: 5.3, 10.7, 14.6, 17.2 and 17.6±0.2 degrees two theta; an X-ray powder diffraction having peaks at about: 5.3, 10.7, 21.6, 25.1 and 27.1±0.2 degrees two theta; a PXRD spectrum as depicted in FIG. 3; and a combination thereof.

33. The crystalline ODV succinate of claim 32, characterized by a PXRD spectrum as depicted in FIG. 3.

34. The crystalline ODV succinate of claim 32, characterized by an X-ray powder diffraction having peaks at about 5.3, 10.7, 14.6, 17.2, 17.6, 21.6, 25.1, and 27.1±0.2 degrees two theta.

35. A method of preparing the crystalline form of O-desmethylvenlafaxine succinate of claim 32 comprising: combining O-desmethylvenlafaxine, a C₁₋₄ alkyl alcohol, a C₆₋₈ aliphatic or aromatic hydrocarbon and succinic acid to form a reaction mixture to precepitate the crystalline ODV succinate from the reaction mixture.

36. The method of claim 35, wherein the C₁₋₄ alkyl alcohol is ethanol, and the C₆₋₈ aliphatic or aromatic hydrocarbon is hexane or toluene.

37. The method of claim 35, wherein the O-desmethylvenlafaxine starting material is a ODV base.

38. The method of claim 35, wherein the ratio of O-desmethylvenlafaxine succinate to the C₁₋₄ alkyl alcohol about 1:2 to about 2:1.

39. The method of claim 38, wherein the ration is about 1:2 (w/v).

40. The method of claim 36, wherein the ratio of ethanol to toluene is about 1:10 to about 1:4 (v/v).

41. The method of claim 40, wherein the ratio is about 2:9.

42. The method of claim 35, wherein the reagents of the reaction mixture are mixed to form a solution.

43. The method of claim 42, wherein the reaction mixture is heated to reflux to obtain a solution.

44. The method of claim 43, wherein the solution is then cooled to obtain the crystalline ODV succinate.

45. The method of claim 35, comprising a) suspending ODV base in ethanol at about 70° C. to about reflux temperature; b) adding succinic acid to the suspension to obtain a solution; c) adding hexane or toluene to the solution; and d) cooling the solution to about 15° C.-30° C.

46. The method of claim 45, wherein ODV base in ethanol is suspended at about reflux temperature.

47. The method of claim 45, wherein hexane or toluene is added to the solution in a drop wise manner.

48. The method of claim 45, wherein the solution is cooled to about room temperature.

49. The method of claim 45, wherein cooling the solution is carried out for a sufficient period of time to obtain the ODV succinate crystalline form.

50. The method of claim 49, wherein cooling the solution is for a period of about 12 hours to about 36 hours.

51. The method of claim 50, wherein cooling the solution is for a period of about 16 hours.

52. A pharmaceutical, composition comprising any of the crystalline O-desmethylvenlafaxine succinate forms of claims 1 or 32, and at least one pharmaceutically acceptable excipient.

53. A process for preparing a pharmaceutical composition comprising any of the crystalline O-desmethylvenlafaxine succinate forms of claims 1 or 32, comprising combining any of the crystalline O-desmethylvenlafaxine succinate forms of claims 1 or 32 with at least one pharmaceutically acceptable excipient.

54. A method of inhibiting re-uptake of norepinephrine and serotonin in a patient comprising administering to a patient in need thereof a therapeutically effective amount of any one of the crystalline O-desmethylvenlafaxine succinate forms of claims 1 or 32.

55. A method of treating a patient comprising administering to a patient in need thereof a therapeutically effective amount of any one of the crystalline O-desmethylvenlafaxine succinate forms of claims 1 or 32.
